(19) United States
(12) Patent Application Publication
(10) Pub. No.: US 2003/0166605 A1
(43) Pub. Date: Sep. 4, 2003

(54) Method of minimizing damage to heart tissue during cardiac surgery and cardiac transplantation

(76) Inventors: Edward Leung, Cary, NC (US); Roger D. Blevins, Apex, NC (US); Robert M. Mentzer JR., Lexington, KY (US); Victor Molina-Viamonte, Buenos Aires (AR)

Correspondence Address: EDWARDS & ANGELL, LLP, P.O. BOX 9169, BOSTON, MA, 02209, US

(21) Appl. No.: 10/313,896

(22) Filed: Dec. 6, 2002

Related U.S. Application Data
(63) Continuation of application No. 09/300,128, filed on Apr. 27, 1999, now abandoned.

(51) Int. Cl. A61K 31/7076; A01N 1/02

(52) U.S. Cl. 514/46; 435/1.1

(57) ABSTRACT

Methods for minimizing ischemic damage and/or reperfusion injury to heart tissue during cardiac surgery where the heart is removed from the body and then re-implanted into the same body, as well as cardiac transplantation, where the heart is removed from one body and transplanted into another body, are disclosed. Prior to removing the heart from the body, adenosine or adenosine A₁ or A₃ receptor agonists can be administered to the patient in a manner which provides cardioprotection to the heart. When the heart is removed from the body, it can be stored in a cardioplegic solution which contains adenosine, hypoxanthine and/or adenosine A₁ or A₃ receptor agonists. After the heart is reimplanted or transplanted, reperfusion injury can be minimized by administering adenosine or adenosine A₂ receptor agonists to the patient in a manner which minimizes reperfusion injury. Preferably, all three steps are taken in order to minimize the amount of ischemic damage and reperfusion injury to the heart.

### FIELD OF THE INVENTION

This invention is generally in the area of minimizing ischemic damage and/or reperfusion injury to heart tissue during cardiac surgery where the heart is removed from the body and then re-implanted into the same body, as well as cardiac transplantation, where the heart is removed from one body and transplanted into another body.

### BACKGROUND OF THE INVENTION

There are many surgical procedures for correcting complex congenital heart abnormalities, placing cardiac valvular prostheses, repairing defective valves, and bypassing obstructed coronary vessels which require the body to be supported with a heart-lung machine while the heart is rendered quiescent by interrupting its blood supply and briefly perfusing it with a cold solution of electrolytes with a relatively high potassium concentration (known as a cardioplegic solution). Placing the heart in a cardioplegic solution allows the surgeon to perform intricate surgical procedures on the heart without the distraction of having the heart pumping while the surgeon is operating, and the absence of blood also allows the surgeon to see more clearly.

When the heart is placed in cardioplegic solution, the surgeon only has a limited amount of time to perform the surgery before irreversible ischemic damage is incurred. That amount of time is approximately 20 to 30 minutes. With the onset of ischemia, the supply of substrates for energy production ceases, and the high energy phosphate adenosine triphosphate (ATP) (which provides energy for contraction and operation of ion pumps in the myocardial cell) is degraded over time to its precursors ADP and AMP. AMP can undergo further degradation at the myocardial membrane to the diffusable purine nucleoside adenosine. Adenosine is also rapidly metabolized to inosine, hypoxanthine and xanthine. With the restoration of blood flow, these nucleosides are washed out of the heart via the circulation.

When a heart is ischemic for a sufficient amount of time, the level of ATP is reduced, and the heart has less energy available for contraction and maintenance of ionic fluxes. Accordingly, the contractile function of the heart may be diminished or lost. Several methods have been developed to extend the length of time a heart can tolerate ischemia, and therefore reduce the morbidity and mortality of cardiac operations.

One method involves using hyperkalemic solutions along with hypothermia to lower the basal metabolic rate of the cardiac tissue. This reduces the rate of ATP degradation during ischemia and increases the amount of time available to the surgeon to perform intricate surgical procedures during surgery. A disadvantage associated with this method is that inadequate myocardial protection during prolonged ischemia may lead to prolonged weaning from the cardiopulmonary bypass machine, the use of inotropic drugs to support the failing heart postoperatively, and the increase in mortality associated with postoperative arrhythmias and/or cardiac failure.

U.S. Pat. No. 4,880,783 to Mentzer et al. discloses that the ability of the myocardium to tolerate ischemia can be enhanced by adding adenosine, hypoxanthine and ribose to standard cardioplegia solutions. The improved effect is purportedly due to the greater preservation of high energy phosphates during ischemia, more rapid recovery of high energy phosphates after ischemia, and a greater recovery of contractile function following an ischemic period. The use of the cardioplegic solution purportedly provides increased protection of the heart during ischemia incurred during surgery, or during the transportation of the heart between donor and recipient for cardiac transplantation.

It would be advantageous to provide additional methods for protecting the heart from irreversible ischemia during cardiac surgery or heart transplantation operations. The present invention provides such methods.

### SUMMARY OF THE INVENTION

Methods for minimizing ischemic damage and/or reperfusion injury to heart tissue during cardiac surgery, for example, where the heart is removed from the body and then re-implanted into the same body, as well as cardiac transplantation, where the heart is removed from one body and transplanted into another body, are disclosed.

Prior to removing the heart from the body, adenosine, adenosine A₁ or adenosine A₃ receptor agonists can be administered to the patient in a manner which provides cardioprotection to the heart. When the heart is removed from the body, it can be stored in a cardioplegic solution which contains adenosine, hypoxanthine and/or adenosine A₁ or A₃ receptor agonists. After the heart is reimplanted or transplanted, reperfusion injury can be minimize by administering adenosine or adenosine A₂ receptor agonists to the patient in a manner which minimizes reperfusion injury.

Preferably, all three steps are taken in order to minimize the amount of ischemic damage and reperfusion injury to the heart.

### BRIEF DESCRIPTION OF THE FIGURES

FIG. 1 is a bar graph representing the percentage of patients receiving high dose dopamine during the clinical trial described in Example 1. The white bar represents the placebo group, the black bar represents the low dose adenosine group, and the checkered bar represents the high dose adenosine group.

FIG. 2 is a bar graph representing the percentage of patients receiving epinephrine during the clinical trial described in Example 1. The white bar represents the placebo group, the black bar represents the low dose adenosine group, and the checkered bar represents the high dose adenosine group.

FIG. 3 is a bar graph representing the percentage of patients suffering from myocardial infarction during the clinical trial described in Example 1. The white bar represents the placebo group, the black bar represents the low dose adenosine group, and the checkered bar represents the high dose adenosine group.

FIG. 4 is a bar graph representing the percentage of patients suffering from mortality during the clinical trial described in Example 1. The white bar represents the placebo group, the black bar represents the low dose adenosine group, and the checkered bar represents the high dose adenosine group.

FIG. 5 is a bar graph representing the percentage of patients suffering from adverse events (high dose dopamine, epinephrine use, insertion of intraaortic balloon pump, myocardial infarction or death) during the clinical trial described in Example 1. The white bar represents the placebo group, the black bar represents the low dose adenosine group, and the checkered bar represents the high dose adenosine group.

### DETAILED DESCRIPTION OF THE INVENTION

Methods for minimizing ischemic damage and/or reperfusion injury to heart tissue during cardiac surgery, for example, where the heart is removed from the body and then re-implanted into the same body, as well as cardiac transplantation, where the heart is removed from one body and transplanted into another body, are disclosed.

Adverse effects associated with cardiac surgery, for example, high-dose dopamine, epinephrine use, insertion of intra-aortic balloon pumps, myocardial infarction and death can be effectively minimed using the methods described herein.

I. Compositions

A. Adenosine Receptor Agonists

Adenosine (Ado) is an autocoid (or local hormone) that modulates numerous functions in the cardiovascular and other organ systems. The actions of Ado are mediated by at least four subtypes of cell surface receptors called A₁, A₂ₐ, A_{2b}, and A₃. Numerous selective adenosine receptor agonists are known.

Because of the ubiquity of adenosine receptors (AdoRs) throughout the human body, their indiscriminate activation may cause undesirable side effects. Therefore, it can be advantageous to administer selective adenosine receptor agonists when the particular receptor to be agonized is known.

As used herein, the term adenosine A₁ receptor agonist is used to define a compound which is selective for the adenosine A₁ receptor, with an affinity for the adenosine A₁ receptor at least 10, and preferably, at least 50 times higher than the affinity for the adenosine A₂ and A₃ receptors.

As used herein, the term adenosine A₂ receptor agonist is used to define a compound which is selective for the adenosine A₂ receptor, with an affinity for the adenosine A₂ receptor at least 10, and preferably, at least 50 times higher than the affinity for the adenosine A₁ and A₃ receptors.

As used herein, the term adenosine A₃ receptor agonist is used to define a compound which is selective for the adenosine A₁ receptor, with an affinity for the adenosine A₁ receptor at least 10, and preferably, at least 50 times higher than the affinity for the adenosine A₁ and A₂ receptors.

Specific and non-specific A₁, A₂ and A₃ receptor agonists are well known to those of skill in the art. Examples of these agonists are found, for example, in the 1999 RBI (Sigma) and Tocris catalogs. Examples of suitable agonists include AB-MECA (A₃), adenosine amine congener (ADAC) (A₁), N⁶-2-(4-aminophenyl)ethyladenosine (APNEA) (A₃), CGS-21680 HCl (A₂ₐ), 2-chloroadenosine (A₁>A₂), 2-chlorocyclopentyladenosine (A₁), N⁶-cyclohexyladenosine (A₁), N⁶-cyclopentyladenosine (A₁), 5′-N-cyclopropyl)-carboxamidoadenosine (A₂), DPMA (PD 125,944) (A₂ₐ), ENBA (S⁻) (A₁), 5′-N-ethylcarboxamidoadenosine (NECA) (A_{2b}), IB-MECA (A₃), MECA (A₂>A₁), 1-methylisoguanosine (A₁), metrifudil (A₂), 2-phenylaminoadenosine (A₂>A₁), N⁶-phenyladenosine (A₁>A₂), N⁶-phenylethyladenosine (A₁>A₂), R-PIA (A₁), S-PIA (A₁), N⁶-sulfophenyladenosine (A¹), and 2-chloro-IB-MECA (A₃).

Since the pharmacology at the adenosine receptors varies between species, especially between rodent and human receptors, it is important to determine the selectivity of the compounds in human adenosine receptors.

Adenosine and adenosine A₂ receptor agonists are effective at minimizing reperfusion injury. Adenosine and Adenosine A₁ and A₃ receptor agonists are primarily responsible for providing cardioprotection when they are administered prior to placing the heart in the cardioplegic solution.

Unlike A₁ and A₃ receptor agonists, A₂ agonists are not believed to be responsible for a significant cardioprotective effect when given prior to placing the heart in the cardioplegic solution. However, when given after the heart is removed from the solution and re-implanted or transplanted and then reperfused, they do provide significant protection against reperfusion injury.

While not wishing to be bound by a particular theory, it is believed that the protection against reperfusion injury is due to an anti-inflammatory effect that stimulation of adenosine A₂ receptors has on heart tissue. It is also believed that adenosine is effective at protecting the reversibly injured heart when administered before ischemia, most likely due to activation of the adenosine A₁ and A₃ receptors in the cardiac myocytes and circulating pro-inflammatory cell types such as mast cells and other leucocytes.

Although selective A₁ and A₃ agonists are preferred for cardioprotection and selective A₂ agonists are preferred for minimizing reperfusion injury, non-selective agonists can be used, and A₂ agonists can be used for cardioprotection and A₁ and A₃ agonists can be used to provide some degree of minimization of reperfusion injury.

The effectiveness of adenosine in reducing reperfusion injury related to treatment of myocardial infarction with thrombolytic agents is known. However, the effect of adenosine or adenosine A₂ receptor agonists at reducing reperfusion injury during cardiac surgery or following transplantation of a heart placed in a cardioplegic solution including adenosine or adenosine A₁ receptor agonists has not been disclosed in any prior art Applicants are aware of.

Adenosine has a relatively short half life (on the order of about 30 seconds), and is typically administered via intravenous or intracoronary injection. Useful dosages for providing cardioprotection prior to placing the heart in cardioplegic solution range from between 10 and 200 μg/kg/min, and are preferably between 40 and 150 μg/kg/min. The same dosages are also useful in minimizing reperfusion injury after the heart has been re-implanted or transplanted, although a dose of between 50 and 70 μg/kg/min may be preferred.

The selective agonists typically have longer half lives, and can be administered via any medically acceptable means. Suitable means of administration include oral, rectal, topical or parenteral (including subcutaneous, intramuscular and intravenous) administration, although oral or parenteral administration are preferred.

The amount of the compound required will, of course, vary with the individual being treated, the binding affinity of the compound for the particular adenosine receptor, and the half-life of the compound in vivo. The amount of the compound to be administered can be readily determined by those of skill in the art by analogy to the effective dosage of adenosine described above. Correlations between effective dosages of adenosine and selective agonists for particular indications has been routinely performed by those of skill in the art.

The dosage is ultimately at the discretion of the medical practitioner. However, a suitable effective dose is one which effectively provides a plasma concentration of about 0.1 μg/kg to about 150 μg/kg. Dosages above or below the range cited above are within the scope of the present invention and may be administered to the individual patient if desired and necessary.

The adenosine or selective adenosine receptor agonists described above are preferably administered in a formulation that includes an acceptable carrier for the mode of administration. Suitable pharmaceutically acceptable carriers are known to those of skill in the art. The formulations can optionally include other therapeutically active ingredients, such as antibiotics, antivirals, healing promotion agents, anti-inflammatory agents, immunosuppressants, growth factors, anti-metabolites, cell adhesion molecules (CAMs), antibodies, vascularizing agents, anti-coagulants, and anesthetics/analgesics.

The carrier must be pharmaceutically acceptable in the sense of being compatible with the other ingredients of the formulation and not deleterious to the recipient thereof. The formulations can include carriers suitable for oral, rectal, topical or parenteral (including subcutaneous, intramuscular and intravenous) administration. Preferred carriers are those suitable for oral or parenteral administration.

Formulations suitable for parenteral administration conveniently include sterile aqueous preparation of the active compound which is preferably isotonic with the blood of the recipient. Thus, such formulations may conveniently contain distilled water, 5% dextrose in distilled water or saline. Useful formulations also include concentrated solutions or solids containing the adenosine or adenosine agonists which upon dilution with an appropriate solvent give a solution suitable for parental administration above.

For enteral administration, the selective agonists can be incorporated into an inert carrier in discrete units such as capsules, cachets, tablets or lozenges, each containing a predetermined amount of the active compound; as a powder or granules; or a suspension or solution in an aqueous liquid or non-aqueous liquid, e.g., a syrup, an elixir, an emulsion or a draught. Suitable carriers may be starches or sugars and include lubricants, flavorings, binders, and other materials of the same nature.

A tablet may be made by compression or molding, optionally with one or more accessory ingredients. Compressed tablets may be prepared by compressing in a suitable machine the active compound in a free-flowing form, e.g., a powder or granules, optionally mixed with accessory ingredients, e.g., binders, lubricants, inert diluents, surface active or dispersing agents. Molded tablets may be made by molding in a suitable machine, a mixture of the powdered active compound with any suitable carrier.

A syrup or suspension may be made by adding the active compound to a concentrated, aqueous solution of a sugar, e.g., sucrose, to which may also be added any accessory ingredients. Such accessory ingredients may include flavoring, an agent to retard crystallization of the sugar or an agent to increase the solubility of any other ingredient, e.g., as a polyhydric alcohol, for example, glycerol or sorbitol.

In addition to the aforementioned ingredients, the formulations may further include one or more optional accessory ingredient(s) utilized in the art of pharmaceutical formulations, e.g., diluents, buffers, flavoring agents, binders, surface active agents, thickeners, lubricants, suspending agents, preservatives (including antioxidants) and the like.

B. Cardioplegic Solutions

Cardioplegic solutions are well known to those of skill in the art. Preferably, the cardioplegic solution includes adenosine, a selective adenosine A₁ or A₃ receptor agonist, hypoxanthine or ribose. Suitable cardioplegic solutions are described in U.S. Pat. No. 4,880,783 to Mentzer et al., the contents of which are hereby incorporated by reference.

Adenosine, hypoxanthine and ribose are endogenous substances. Adenosine and hypoxanthine are purine nucleosides and ribose is a sugar. When these substances are used as additives in conventional cardioplegic solutions, a relatively high local concentration in the heart can be achieved, without exposure to the systemic circulation. Following the re-implantation or transplantation of the heart, these substances are washed out of the myocardium and rapidly distributed and metabolized.

These substances facilitate the preservation and repletion of the adenine nucleotide pool during ischemia by serving as substrate for the purine nucleotide salvage pathways. During ischemia, the intracellular adenine nucleotide pool is degraded to the diffusable nucleosides adenosine, inosine and hypoxanthine. These nucleosides are then washed out during the reperfusion period. ATP levels may be depressed for as long as 7-10 days due to the loss of these nucleotide precursors adenosine, inosine, hypoxanthine.

When present in a cardioplegia solution, adenosine and hypoxanthine may be capable of preserving and/or restoring myocardial levels of ATP. Either adenosine or hypoxanthine may help to restore the contractile function of the isolated perfused rat heart after a period of ischemia A cardioplegia solution supplemented with adenosine or hypoxanthine may reduce the rate of ATP degradation during ischemia. It may also prevent the leakage of adenosine or hypoxanthine from the heart cells by decreasing the concentration gradient of adenosine or hypoxanthine across the cell membrane.

In one embodiment, the cardioplegic solution includes adenosine in a concentration of about 100 μmoles per liter, hypoxanthine in a final concentration in the solution of about 100 μmoles per liter, and/or ribose in a final concentration in the solution of about 2 mmoles per liter. The electrolytes include Na, Cl, K, Ca and Mg ions in solution in the following approximate concentrations:

| | | |
|---|---|---|
| Na⁺ | 110 | meq/l |
| Cl⁻ | 160 | meq/l |
| K⁺ | 16 | meq/l |
| Ca⁺⁺ | 2.4 | meq/l |
| 5 Mg⁺⁺ | 32 | meq, and |

NaHCO₃ or HCl to adjust pH to 7.4.

II. Methods

A. Methods of Performing Cardiac Surgery

In performing cardiac surgery, before placing the heart in a cardioplegic solution, adenosine, an adenosine A₁ receptor agonist or an adenosine A₃ receptor agonist are administered to the patient in a manner which provides cardioprotection to the heart. Effective dosages rate for adenosine and adenosine A₁ and A₃ receptor agonists have been previously described. Suitable durations of the adenosine administration are between 10 minutes and 1 hour, although longer durations would not be expected to adversely effect the patient.

Following administration of the adenosine or adenosine A₁ or A₃ agonists, the heart is placed in a cardioplegic solution, and the surgeon performs the necessary surgical operation. When the operation is complete, the heart is re-implanted into the patient.

Following re-implantation, adenosine or adenosine A₂ receptor agonists are administered to the patient in a manner which minimizes reperfusion injury to the heart. Effective dosages rate for adenosine and adenosine A₂ receptor agonists have been previously described. Suitable durations of the adenosine administration are between 10 minutes and 1 hour, although longer durations, for example, up to three hours, would not be expected to adversely effect the patient.

It is advantageous to cool the body down during surgery. Lowering the body temperature can prolong the amount of time the surgeon has for performing the cardiac surgery. Further, the temperature of the cardioplegic solution is advantageously lowered as well. An additional advantage of cooling the patient's body is that a higher dosage of adenosine may be given without causing hypotensive effects. Further, adenosine is metabolized more slowly when the body temperature is lowered.

Preferably, all three steps are taken in order to minimize the amount of ischemic damage and reperfusion injury to the heart. However, combinations of at least two of the three steps described above will provide an advantage over merely incorporating adenosine or an adenosine A₁ and/or A₃ receptor agonist into the cardioplegic solution.

B. Methods of Performing Heart Transplantation

In performing a heart transplantation, a heart from a brain-dead individual is removed, placed in a cardioplegic solution, and transplanted into another individual. The heart is often kept refrigerated in the cardioplegic solution to minimize damage.

Before the heart is removed, the brain-dead patient can be given an effective dosage of adenosine or an adenosine A₁ or A₃ receptor agonist to provide cardioprotection to the heart. This dosage is the same dosage as that provided above during cardiac surgery before the heart is placed in the cardioplegic solution.

When the heart is removed and placed in a cardioplegic solution, the heart is preferably refrigerated until the transplantation. Following transplantation, adenosine or an adenosine A₂ receptor agonist is administered to minimize reperfusion injury. The dosage is the same dosage as that provided above during cardiac surgery after the heart is re-implanted into the patient.

The present invention will be further understood with reference to the following non-limiting examples:

### EXAMPLE 1

Double Blind, Placebo Controlled Trial

A double blind, placebo controlled trial was performed on 253 patients randomized into three groups. The objective of the study was to evaluate the safety, tolerance and efficacy of adenosine in patients undergoing coronary artery bypass surgery. Inadequate myocardial protection in patients undergoing coronary artery bypass surgery contributes to overall hospital mortality and morbidity.

The treatments included the intraoperative administration of cold blood cardioplegia, blood cardioplegia including 500 μM adenosine (“low dose adenosine”), and blood cardioplegia including 200 mM adenosine (“high dose adenosine”). Patients receiving adenosine were also given an infusion of adenosine (200 μg/kg/min) 10 minutes before and 15 minutes after removal of the aortic crossclamp. Invasive and non-invasive measurements of ventricular performance were obtained before, during and after surgery.

Results: The high dose adenosine group was associated with a trend toward a decrease in high-dose dopamine support and a lower incidence of myocardial infarction. A composite outcome analysis demonstrated that patients who received high dose adenosine were less likely to experience one of five adverse events: high dose dopamine use, epinephrine use, insertion of intra-aortic balloon pump, myocardial infarction or death. The operative mortality rate for all patients studied was 3.6% (9/253).

Patient Selection: Study patients included those who were electively scheduled for coronary artery bypass surgery and had an ejection fraction of less than or equal to 0.40. Exclusion criteria included known or suspected pregnancy, known hypersensitivity to adenosine, and enrollment in another clinical trial study.

Study Design: 253 patients were split into three groups. Group A patients (n=84) received placebo and were given standard hyperkalemic cold blood cardioplegia. Group B patients (low dose adenosine, n=84) were given hyperkalemic cold blood cardioplegia containing 500 μM adenosine. Group C patients (high dose adenosine) were given hyperkalemic cold blood cardioplegia containing 200 mM adenosine. The patients who received adenosine cardioplegia were also exposed to a 10 minute infusion of adenosine pre-treatment (200 μg/kg/min) immediately before application of the aortic crossclamp and a 15 minute infusion of adenosine immediately after removing the crossclamp.

Before surgery, patients were evaluated for the degree of ischemic disease by history, echocardiography, and cardiac catheterization. In the operating room, hemodynamic measurements were obtained and recorded just before initiating cardiopulmonary bypass and 15, 30, 45 and 60 minutes and 2, 3, 4, 5, and 8 hours after cessation of bypass. In patients requiring intravenous inotropic medications in the postoperative period, hemodynamic monitoring was continued with measurements of specified parameters every 2 hours for 24 hours, and then every 4 hours until the inotropic medications were discontinued or it was ascertained that monitoring was no longer helpful in the management of the patient.

Invasive hemodynamic measurements included systolic blood pressure, heart rate, central venous pressure, pulmonary artery pressure, pulmonary capillary wedge pressure and cardiac output. The cardiac index, stroke volume, systemic vascular resistance, pulmonary vascular resistance, right ventricular stroke work index and left ventricular stroke index were derived. The cardiac output measurements were obtained using a thermodilution catheter and computer. Noninvasive heart function studies included 12-lead electrocardiograms, preoperative stress dobutamine echocardiography, and pre- and post-operative transthoracic and transesophageal echocardiograms. Patients were monitored from the time of enrollment to follow-up 4 to 6 weeks after discharge from the hospital. This included routine blood work and chemistries, arterial blood gases, pH, creatine kinase (CK)-MB concentrations, and pulse oximetry.

Outcomes:

The primary endpoints of the study were reduction in total dopamine use during the first 7 days, reduction in all inotropic support required during the first 7 days, reduction in the use of dopamine to less than 5 μg/kg/min.

There were 21 secondary endpoints, including improvement in postoperative hemodynamics, reduction in the use of the intraaortic balloon pump, reduction in the incidence of myocardial infarction, and decrease in the mortality rate. Diagnosis of MI required the confirmation of two of the following criteria: 12 lead electrocardiogram with new and persistent Q waves, CK-MB greater than 30 IU/L or >5.0 ng/ml, CK index>2.7 and echocardiography demonstrating new wall motion abnormalities.

Data Analysis:

All patients who received study treatment and underwent coronary bypass surgery were included in the intent-to-treat analysis. Categorical primary and secondary endpoints were analyzed using the Pearson chi square test, comparing the percentage of patients in the placebo group to the low and high dose adenosine groups. The continuous hemodynamic profiles were analyzed using a repeated measures analysis to assess the rate of change from baseline values. To take into account the baseline values for each patient, the percentage change from baseline was computed for each hemodynamic outcome. The hemodynamic outcomes were heart rate, systolic blood pressure, cardiac index, pulmonary capillary wedge pressure, central venous pressure, pulmonary artery pressure, left ventricular stroke index, and right ventricular stroke work index. A repeated measures analysis was used to analyze the percentage change from baseline of these outcomes over the first 24 hours off cardiopulmonary bypass for each treatment. In the statistical model, the interaction of time and treatment tested whether the slopes of the lines that pass through the time points of each treatment were significantly different from one another at a level of 5%. When the time by treatment interaction was significant, a statistical comparison of each pair of treatment slopes was performed using the least significant difference pairwise procedure. The slopes were interpreted as an increase or decrease in the percentage change of the hemodynamic outcomes from baseline over time. A compound symmetry structure was used to model the covariances and variances of the time points. All statistical testing was performed with SAS software.

Results: Two hundred and fifty three patients were enrolled and completed the study. The medical history (e.g., incidence of congestive heart failure, angina, arrhythmias, prior MI, previous coronary artery angioplasty, and previous coronary artery bypass surgery) was similar among the three treatment groups. Likewise, there were no differences with respect to mean age, gender, ejection fraction, crossclamp time, cardiopulmonary bypass time, preoperative hemoglobin levels and platelet counts. The total duration of cardioplegia and the total volume of cardioplegia administered to the patients was also similar.

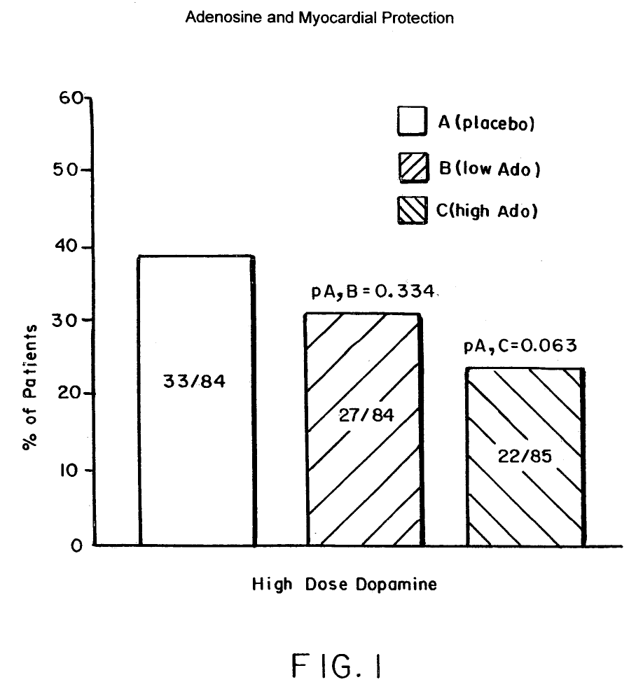

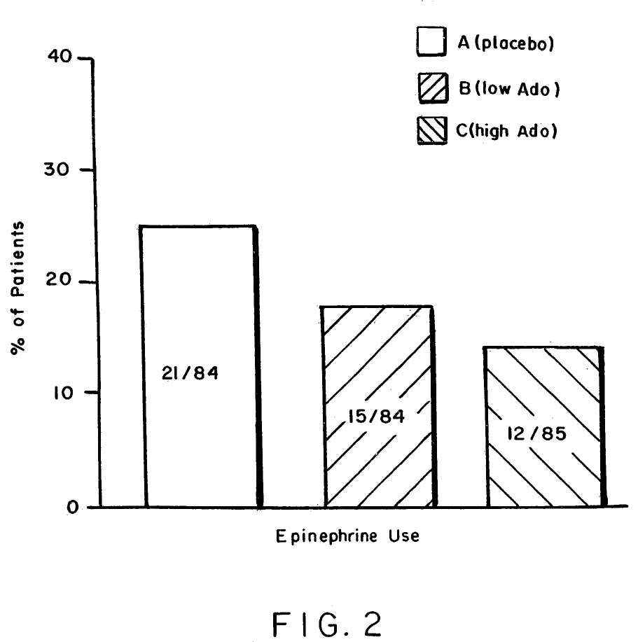

In the first 7 days after surgery, 77% of the patients in the placebo group, 71% of the patients in the low dose adenosine group and 79% of the patients in the high dose adenosine group received dopamine. There was no significant difference between the placebo and either the high or low dose adenosine groups. Likewise, the use of any inotropic agent (dopamine, miltinone, amrinone, epinephrine, norepinephrine, dobutamine or isoproterenol) during the first 7 days after surgery was similar (79, 73 and 80%, respectively). There was a trend toward a reduction in the number of patients requiring high dose dopamine (>5 μg/kg/min) as shown in FIG. 1, and intravenous epinephrine, as shown in FIG. 2.

There was no significant time by treatment interaction for the hemodynamic variables of central venous pressure, pulmonary artery pressure, left ventricular stroke index, or right ventricular stroke work index. The results for the other hemodynamic parameters are shown in Table 1. The percentage change in systolic blood pressure from baseline was not affected by treatment. With respect to heart rate, there was a significant time by treatment interaction (p=0.004). Pairwise comparison showed that patients in the placebo group were significantly different from the low- and high-dose adenosine groups. Although the absolute mean heart rate was similar among all three groups at baseline (69.6±1.7, 66.5±1.6, and 67.4±1.7 beats per minute, respectively) and 24 hours after surgery (95.1±3.4, 90.7±3.6, and 96.8±5.4 beats per minute, respectively), stabilization of the heart rate was achieved sooner in patients receiving high dose adenosine.

TABLE 1
Effect of Adenosine Treatment on the Percentage Change in
Selected Hemodynamic Variables Compared With Baseline
During the First 24 Postoperative Hours

| Variable | Group | Slope ± SE | Comparison | p Value |
|---|---|---|---|---|
| Heart Rate | A | 0.254 ± 0.079 | A vs. B | 0.0216 |
| | B | 0.510 ± 0.078 | B vs. C | NS |
| | C | 0.820 ± 0.081 | A vs. C | 0.0013 |
| Systolic Blood | A | 0.532 ± 0.061 | A vs. B | NA |
| Pressure | B | 0.387 ± 0.060 | B vs. C | NA |
| | C | 0.440 ± 0.063 | A vs. C | NA |
| Cardiac Index | A | 0.983 ± 0.109 | A vs. B | NS |
| | B | 1.130 ± 0.105 | B vs. C | 0.0277 |
| | C | 1.468 ± 0.112 | A vs. C | 0.0020 |
| Pulmonary capillary | A | 0.523 ± 0.272 | A vs. B | NS |
| wedge pressure | B | 0.688 ± 0.249 | B vs. C | 0.0001 |
| | C | −0.869 ± 0.288 | A vs. C | 0.0005 |

NA = Not appropriate
NS = Not significant
Data are presented as the mean slope of the line ± SE for each outcome in each treatment. The slopes are interpreted as the increase or decrease of the percentage of the hemodynamic outcome from baseline for every hour of time during the first 25 hours off bypass.

As reflected by the slopes in Table 1, the cardiac index improved more rapidly in patients receiving high dose adenosine versus placebo treatment (p=0.002). Normalization of pulmonary capillary wedge pressure also occurred more rapidly in the patients receiving high dose adenosine.

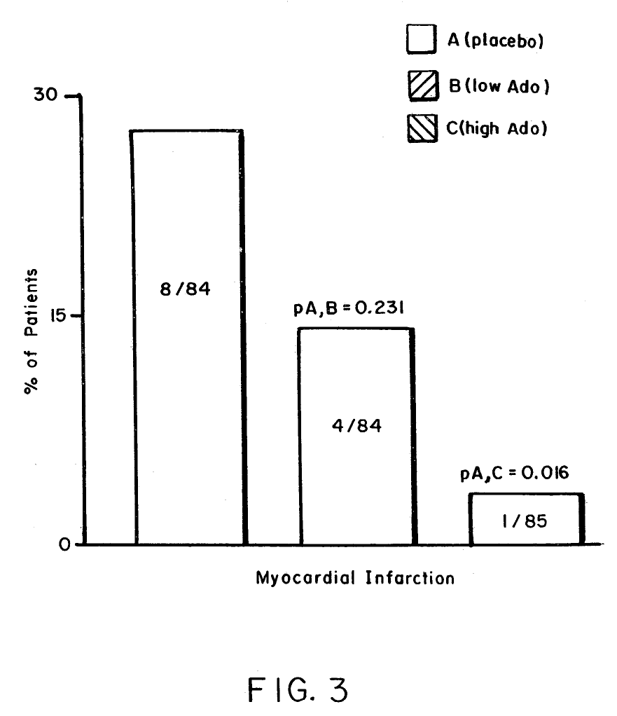

Overall, 6.3% of the patients required insertion of an intraaortic balloon pump for low cardiac output. There were nine insertions in the placebo group, two in the low dose adenosine group, and five in the high dose adenosine group. The overall incidence of postoperative MI was relatively low (5.1%). Nevertheless, the MI rate in the high dose adenosine group was lower when compared with the placebo group (1.2% v. 9.5%), as shown in FIG. 3.

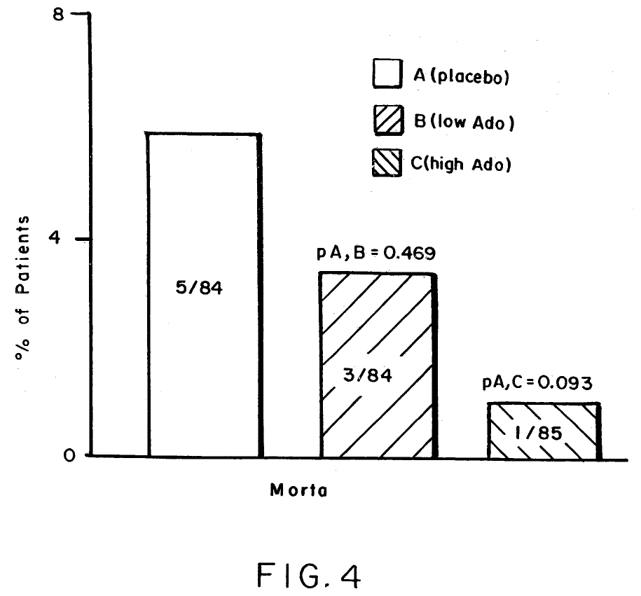

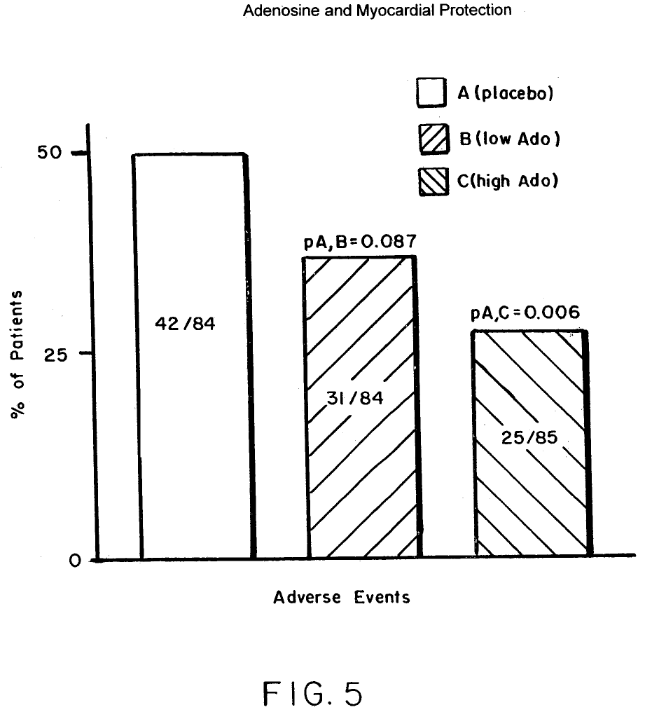

The overall death rate for the entire study population was 3.6%. There was a trend toward a lower rate in the adenosine-treated patients versus the placebo group (1.2%, 3.6% and 6.0% for high dose adenosine, low dose adenosine, and placebo, respectively), as shown in FIG. 4. When a composite outcome of high dose dopamine, epinephrine use, insertion of the intraaortic balloon pump, MI and death was analyzed (as shown in FIG. 5), the percentage of patients experiencing one of these adverse events was lower in patients treated with high dose adenosine (p=0.006).

The administration of high dose adenosine in patients undergoing coronary artery bypass surgery using cardiopulmonary bypass is safe and well tolerated. The use of adenosine appears to be associated with improved postoperative hemodynamic function. Adenosine treatment appears to be associated with a decrease in mortality and morbidity.

1. A method for minimizing ischemic damage to a heart during cardiac surgery or heart transplantation, comprising:
a) administering an effective amount of adenosine or an adenosine A₁ or A₃ receptor agonist prior to placing the heart in cardioplegic solution,
b) placing the heart in a cardioplegic solution,
c) optionally performing cardiac surgery on the heart,
d) re-attaching or transplanting the heart, and
e) administering an effective amount of adenosine or an adenosine A₂ receptor agonist to minimize reperfusion injury.

2. The method of claim 1, wherein the adenosine is administered at a dosage rate of between 50 and 200 μg/kg/min for a period of time between 10 minutes and 4 hours

3. The method of claim 1, wherein the adenosine is administered at a dosage of between 40 and 200 μg/kg/min for a period of time between 5 minutes and 4 hours.

4. The method of claim 1, wherein the cardioplegic solution comprises adenosine or an adenosine A₁ or A₃ receptor agonist.

5. The method of claim 1, wherein the patient is administered an effective reperfusion injury reducing amount of adenosine or an adenosine A₂ receptor agonist following re-implantation or transplantation of the heart.

6. A method for minimizing ischemic damage to a heart during cardiac surgery or heart transplantation comprising administering an effective amount of adenosine or an adenosine A₁ or A₃ receptor agonist prior to placing the heart in cardioplegic solution.

7. The method of claim 6, wherein the adenosine is administered at a dosage of between 50 and 200 μg/kg/min for a period of time between 5 minutes and 4 hours.

8. The method of claim 6, wherein the adenosine is administered at a dosage of between 50 and 140 μg/kg/min for a period of time between 10 and 30 minutes.

9. The method of claim 6, wherein the cardioplegic solution comprises adenosine or an adenosine A₁ or A₃ receptor agonist.

10. The method of claim 6, wherein the patient is administered an effective reperfusion injury reducing amount of adenosine or an adenosine A₂ receptor agonist following re-implantation or transplantation of the heart.

11. A method for minimizing ischemic damage to a heart during cardiac surgery or heart transplantation comprising administering an effective amount of adenosine or an adenosine A₂ receptor agonist following re-implantation or transplantation of the heart.

12. The method of claim 11, wherein the adenosine is administered at a dosage of between 40 and 200 μg/kg/min for a period of time between 5 minutes and 4 hours.

13. The method of claim 11, wherein the adenosine is administered at a dosage of between 50 and 140 μg/kg/min for a period of time between 10 and 30 minutes.

14. The method of claim 11, wherein the cardioplegic solution the heart is placed in during the cardiac surgery or the transplantation surgery comprises adenosine or an adenosine A₁ or A₃ receptor agonist.

15. The method of claim 11, wherein the patient from whom the heart is removed and placed in cardioplegic solution is administered an effective cardioprotective amount of adenosine or an adenosine A₁ or A₃ receptor agonist before the heart is placed in the cardioplegic solution.
